(19) United States
(12) Patent Application Publication
(10) Pub. No.: US 2023/0374158 A1
(43) Pub. Date: Nov. 23, 2023

(54) BISPECIFIC MOLECULES AND METHODS OF TREATMENT USING THE SAME

(71) Applicant: ALMIRALL, S.A., Barcelona (ES)

(72) Inventors: Edward HSIA, Barcelona (ES); Vicente Marco GARCÍA GONZÁLEZ, Barcelona (ES)

(21) Appl. No.: 18/031,271

(22) PCT Filed: Oct. 12, 2021
(86) PCT No.: PCT/EP2021/078195

§ 371 Date: Apr. 12, 2023

(30) Foreign Application Priority Data
Oct. 13, 2020 (EP) 20382897.5

(51) Int. Cl. C07K 16/46

(52) U.S. Cl. CPC C07K 16/468; C07K 2317/31; C07K 2317/24

(57) ABSTRACT

The present invention relates to bispecific antigen binding molecules targeting (i) IL-13 or IL-13R and (ii) OX40L or OX40, pharmaceutical compositions comprising the same, and methods of treatment using the same, e.g. in treating a dermatological disease or condition such as atopic dermatitis.

### FIELD OF THE INVENTION

The present invention relates to bispecific antigen binding molecules targeting (i) IL-13 or IL-13R and (ii) OX40L or OX40, pharmaceutical compositions comprising the same, and methods of treatment using the same, e.g. in treating a dermatological disease or condition such as atopic dermatitis.

### BACKGROUND TO THE INVENTION

Atopic dermatitis (AD) is a chronic, relapsing/remitting, noncontagious, pruritic inflammatory systemic skin disease. It is a common and increasingly prevalent disease and in developed nations it is estimated to affect approximately 15-30% of children and 2-10% of adults, with an estimated 20% of people believe to be affected at some point in their life by AD. Whilst AD may occur at any age, it typically starts in childhood and the disease is more common in children. Although many people outgrow the condition, the disease is nonetheless prevalent in adults where it may occur as a persistent disease from childhood or as adult-onset or recurrent AD. AD is often associated with elevated serum IgE levels and sufferers often have a personal or family history of allergic conditions such as allergic rhinoconjunctivitis, asthma, or food allergies (Boguniewicz et al. (2017), J Allergy Clin Immunol Pract 5(6):1519-1531). AD is associated with an increased risk of infections and of developing serious clinical conditions such as coronary artery disease, ischemic stroke, and other cardiovascular diseases. AD patients are also of increased risk of lymphoma although this may be due to steroid use by AD patients ((Boguniewicz et al. (2017)). Symptoms of AD include erythema, edema, xerosis, erosions/excoriations, oozing and crusting, and lichenification, with pruritus (itching) being considered to be a hallmark of the condition (Kirchhof et al. (2018), J Cutan Med Surg., 22(IS) 6S-9S).

The pathophysiology of AD is considered to be complex with evidence of there being a role for genetic, environmental, and immunologic factors and for a central role of the type 2 inflammatory pathway (Kirchhof et al. (2018)). Unlike psoriasis which is predominantly driven by Th17, AD has a multifactorial pathophysiology and, as such, optimal efficacy may require the targeting of multiple pathways.

Bispecifics offer the potential to target multiple pathways and to provide a competitive advantage compared to other biologics currently in development for AD. Advantages of bispecifics may include superior efficacy to “monads”, and bispecifics may provide comparable or superior PK values (dosing frequency) and comparable or superior safety profiles to “monads”.

Numerous cytokines and other factors have been implicated in AD and antibodies targeting a variety of molecules are currently in clinical trials or development for AD. Examples include anti-IL-1a, anti-IgE, anti-IL-4, anti-IL-4Ra, anti-IL-5, anti-12/23p40, anti-IL-13, anti-13R, anti-17A, anti-17C, anti-IL-22R, anti-IL-23A, anti-IL-31, anti-IL-31Ra, anti-IL-33, anti-IL-33R, anti-OX40L and anti-TSLP antibodies.

T helper type 2 (Th2)-associated cytokines have pleiotropic effects on the innate and adaptive immune system. In synergy with tumour necrosis factor a, IL-4 and IL-13 induce thymic stromal lymphopoietin (TSLP) production in keratinocytes and augment the ongoing Th2 skewing of the immune system. IL-4 and IL-13 downregulate mRNA expression and protein synthesis of several structural barrier proteins including filaggrin, involucrin, and loricrin, thus inducing skin barrier dysfunction and aggravation of keratinocyte-mediated immune activation. Due to the Th2-driven inflammatory characteristics of AD, Th2-related molecules may provide an attractive target in order to reduce inflammation and break the detrimental feedback loop.

However the pathophysiology of AD is complex. Although type-2 mechanisms are dominant, there is increasing evidence that the disorder involves multiple immune pathways. In AD patients, the numbers of OX40L positive dendritic cells (DCs) are highly increased, and also its partner, OX40, is upregulated at the sites of inflammation on infiltrating lymphocytes. Blocking the OX40-OX40L pathway has been shown to be protective in several animal models of human autoimmune diseases such as AD, asthma, irritable bowel disease, transplant rejection, autoimmune diabetes, GvHD, autoimmune encephalomyelitis. Dual IL-13 plus OX-40L blockade will block type 2 response evoked by IL-13, and will have a broad inhibition of the different Th subsets.

Numerous medications and treatments are available for the management of AD. These generally aim at reducing skin inflammation and itching (pruritus), restoring skin barrier function, and improving health-related quality of life (HRQoL). Available therapies include moisturizing and basic care (e.g. trigger avoidance), phytotherapy, topical therapies, and systemic therapies. Examples of medications used in the treatment of AD include anti-itch creams, antihistamines, topical corticosteroids (TCS), and calcineurin inhibitors. In more recent years antibody-based treatments have been developed.

Various drawbacks of available treatment methods have been reported in the art including safety and toxicity concerns, development of resistance to treatment (e.g. resistance to TCSs, or to calcineurin inhibitors), as well as patient tolerability and convenience resulting in poor patient compliance. There thus exists a need for the development of new treatment methods for AD.

Besides AD, IL-13 and OX40L have been identified as important factors in numerous other diseases and conditions, and antagonism of these cytokines may accordingly be useful in the treatment of these diseases and conditions.

Examples of diseases and conditions in which IL-13 is implicated include: dermatological diseases (e.g. atopic dermatitis, prurigo nodularis, chronic hand eczema, allergic dermatitis), asthma, allergic rhinitis, COPD, cancer, inflammatory bowel disease, fibrosis, scleroderma, and eosinophilic esophagitis (see e.g. May R and Fung M. Cytokine 2015;75:89-116, and Gandhi N. et al. Nat. Rev. Drug Discover. 2016;15:35-50).

Examples of diseases and conditions in which OX40L is implicated include: dermatological diseases (e.g. atopic dermatitis, prurigo nodularis, chronic hand eczema, allergic dermatitis), gastrointestinal autoimmune diseases (e.g. ulcerative colitis or Crohn's Disease), allergic encephalitis, graft-vs-host disease, proliferative lupus nephritis, rheumatoid arthritis, inflammatory muscle diseases, inflammatory vasculitis, asthma, and collagen-induced arthritis (see e.g.Murata et al. J Immunol 2002; 169:4628-4636 and Hori, Internat. J. Hematol. 2006; 83:17-22)).

### SUMMARY OF THE INVENTION

The invention provides a bispecific antigen-binding molecule comprising: a first antigen binding domain (B1) which is an IL-13 or IL-13R antigen binding domain and a second antigen binding domain (B2) which is an OX40L or OX40 antigen binding domain and wherein the bispecific antigen-binding molecule specifically binds to (i) IL-13 or IL-13R and (ii) OX40L or OX40.

The bispecific antigen-binding molecule antagonises both IL-13 signalling from IL-13R and OX40L signalling from OX40. The antagonist effect on signalling may be achieved because the bispecific antigen-binding molecule interferes with the interaction between each ligand and each receptor, or may be achieved because the molecule interferes with the receptor multimerisation which follows ligand engagement.

The invention also provides a pharmaceutical composition comprising a bispecific antigen-binding molecule of the invention and a pharmaceutically acceptable carrier.

The invention further provides a bispecific antigen-binding molecule of the invention for use a medicament.

Also provided is a method of treating a disease or condition in a patient, wherein the disease or condition is associated with or mediated by IL-13 and/or OX40L, and wherein the method comprises administering to the patient a bispecific antigen-binding molecule of the invention.

The invention further provides a bispecific antigen-binding molecule of the invention for use in a method of treating a disease or condition in a patient, wherein the disease or condition is associated with or mediated by IL-13 and/or OX40L, and wherein the method comprises administering to the patient a bispecific antigen-binding molecule of the invention.

The invention also provides a bispecific antigen-binding molecule of the invention for use in the manufacture of a medicament for the treatment of a disease or condition in a patient wherein the disease or condition is associated with or mediated by IL-13 and/or OX40L.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, the term “an agent” includes a reference to a single agent as well as a plurality of agents (including mixtures of agents).

As used herein, the term “about” as used in relation to a numerical value means, for example, ±25% of the numerical value, preferably ±15%, more preferably ±10%, more preferably still ±5%, and most preferably ±2% or ±1%. Where necessary, the word “about” may be omitted from the definition of the invention.

The term “polypeptide” is used herein in its broadest sense to refer to a compound of two or more subunit amino acids, amino acid analogues, or other peptidomimetics. The term “polypeptide” therefore includes short peptide sequences, longer polypeptides and proteins. The term “amino acid” may refer to either natural and/or unnatural or synthetic amino acids, including both D or L optical isomers, as well as amino acid analogues and peptidomimetics.

The term “antibody” as referred to herein includes whole antibodies and any antigen binding fragment thereof. Whilst an antibody may assume a variety of forms and characteristics, an antibody typically refers to a glycoprotein comprising at least two heavy (H) chains and two light (L) chains inter-connected by disulfide bonds, or an antigen-binding fragment thereof. Each heavy chain is comprised of a heavy chain variable region (abbreviated herein as HCVR or VH) and a heavy chain constant region. Each light chain is comprised of a light chain variable region (abbreviated herein as LCVR or VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR).

An antibody may comprise or consist of a complete antibody molecule having full length heavy and light chains or an antigen-binding fragment thereof. The term “antigen-binding fragment” or the like of an antibody includes a reference to a portion of an antibody that retains the ability to specifically bind to an antigen. It has been shown that the antigen-binding function of an antibody can be performed by fragments of a full-length antibody. The antibodies and antigen binding fragments thereof may be, but are not limited to Fab, modified Fab, Fab′, modified Fab′, F(ab′)₂, Fv, single chain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). Methods for creating and manufacturing these antibody fragments are well known in the art (see for example Verma et al., 1998, Journal of Immunological Methods, 216, 165-181) and the fragments may be screened for utility in the same manner as intact antibodies. Other antibody fragments for use in the present invention include the Fab and Fab′ fragments described in WO 2005/003169, WO 2005/003170 and WO 2005/003171 and Fab-dAb fragments described in WO 2009/040562. Multivalent antibodies may comprise multiple specificities or may be monospecific (see for example WO 92/22853 and WO 05/113605 and the DVD-Ig as disclosed in WO 2007/024715, or the so-called (FabFv)₂Fc described in WO 2011/030107). An alternative multi-specific antigen-binding fragment comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in WO 2015/197772, which is hereby incorporated by reference in its entirety and particularly with respect to the discussion of antibody fragments. These antibody fragments may be obtained using conventional techniques known to those of skill in the art, and the fragments may be screened for utility in the same manner as intact antibodies.

The term antibody encompasses monoclonal antibodies, polyclonal antibodies, monospecific antibodies, and multispecific (e.g. bispecific) antibodies, as well as antigen-binding fragments thereof. A multispecific antibody is capable of binding to at least two target epitopes, typically on separate antigens. In the case of a bispecific antigen-binding molecule of the invention, the bispecific antigen-binding molecule is capable of binding to two separate antigens ((i) IL-13 or IL-13R and (ii) OX40L or OX40).

The term antibody encompasses antibodies of any class (e.g. an IgG, IgE, IgM, IgD, IgA or IgY antibody) or subclass (e.g. IgA1, IgA2, IgG1, IgG2, IgG3 or IgG4). An antibody may, for instance, be a chimeric antibody, a CDR-grafted antibody, a nanobody, a human or humanised antibody, or an antigen-binding fragment of any of the foregoing. Typically, the antibody is a human antibody or a human antibody derivative. The term “human antibody derivative” and the like includes refers to any modified form of the human antibody, e.g. a conjugate of the antibody and another agent (e.g. a drug) or antibody. Fully human antibodies are those antibodies in which the variable regions and the constant regions (where present) of both the heavy and the light chains are all of human origin, or substantially identical to sequences of human origin, but not necessarily from the same antibody.

The terms “disease”, “disorder” and “condition” may be used herein interchangeably, unless the context clearly dictates otherwise.

All publications, patents and patent applications cited herein, whether supra or infra, are hereby incorporated by reference in their entirety.

### IL-13 and OX40L

The terms “targets” and “directed to” and the like may be used interchangeably herein. The present invention provides bispecific antigen-binding molecules which target (i) IL-13 or IL-13R and (ii) OX40L or OX40. The molecules are useful in the methods of the present invention, e.g. to treat a dermatological disease or condition such as AD. The general class of the molecule of the invention may be referred to herein as “an anti-IL-13/IL-13R & OX40L/0X40 bispecific antigen-binding molecule”.

A bispecific antigen-binding molecule which targets IL-13/IL-13R may specifically bind to IL-13 or IL-13R and a bispecific antigen-binding molecule which targets OX40L/OX40 may specifically bind to OX40L or OX40. Thus, within the general class of molecule of the invention, the following specific embodiments are provided:
(i) a bispecific antigen-binding molecule which specifically binds to IL-13 and OX40L (optionally referred to herein as “an anti-IL-13/OX40L bispecific antigen-binding molecule”);
(ii) a bispecific antigen-binding molecule which specifically binds to IL-13 and OX40 (optionally referred to herein as “an anti-IL-13/OX40 bispecific antigen-binding molecule”);
(iii) a bispecific antigen-binding molecule which specifically binds to IL-13R and OX40L (optionally referred to herein as “an anti-IL-13R/OX40L bispecific antigen-binding molecule”); and
(iv) a bispecific antigen-binding molecule which specifically binds to IL-13R and OX40 (optionally referred to herein as “an anti-IL-13R/OX40 bispecific antigen-binding molecule”).
   Option (i) is particularly preferred.

By binding to at least one partner of each of the IL-13/IL-13R and OX40L/OX40 interactions, the bispecific antigen-binding molecule antagonises both IL-13 and OX40L. The bispecific antigen-binding molecule of the invention is therefore an IL-13 antagonist as well as an OX40L antagonist. The term “antagonist” and the like includes a reference to a substance (e.g. a bispecific antigen-binding molecule of the invention) which inhibits or attenuates one or more biological activities of a ligand molecule (e.g. IL-13 or OX40L), such as intracellular signalling mediated by the ligand molecule when bound to its receptor. An antagonist may inhibit or attenuate the binding or interaction of the ligand with its receptor (by binding to either the target or the receptor), but could also for instance inhibit the dimerization of the receptor without affecting the binding of the ligand to the receptor. In some embodiments, the binding between the ligand and its receptor is completely or substantially blocked. An antagonist may, for instance, be a neutralising antibody.

In some embodiments, an antagonist may inhibit dimerization or multimerisation of the receptor without inhibiting or attenuating the binding or interaction of the ligand with one or more of the receptors. For example, in the case of IL-13 antagonism, the antigen-binding molecule may inhibit IL-13 signalling by inhibiting IL4Ra/IL13Ra1 dimerization without inhibiting the binding of IL13 to IL13Ra1 or IL13Ra2. In some embodiments, the dimerization of the receptor may be completely or substantially inhibited. As another example, in the case of OX40L antagonism, the antigen-binding molecule may inhibit OX40L signalling by inhibiting OX40 multimerization without inhibiting the binding of OX40L to OX40. In some embodiments, the multimerization of the receptor may be completely or substantially inhibited.

The expression “IL-13” (interleukin-13) as used herein includes any native mammalian IL-13 sequence (e.g. human, non-human primate (e.g. monkey) or mouse), preferably human IL-13. The term encompasses full-length, unprocessed IL-13 as well as any form of IL-13 resulting from cellular processing. The term encompasses wild type proteins, naturally occurring variants, e.g. splice variants or allelic variants, as well as any other isoforms and mutant forms, as well as modified and unmodified forms of any of the foregoing. A reference to IL-13 includes proteins which may, for instance, be produced recombinantly or by synthetic methods and which have the same amino acid sequence as a naturally occurring or endogenous mammalian IL-13. Where the corresponding mammal is human, the protein may be referred to as hIL-13. The nucleotide and amino acid sequences of IL-13 from various species have been determined and are readily available from public sequence databases. The term hIL-13 encompasses the exemplary hIL-13 sequences accessible at UniProtKB Accession No. P35225 or as set forth in SEQ ID NO. 1 and SEQ ID NO. 2 (with and without the signal peptide respectively), as well as biologically active fragments thereof and other hIL-13 sequences that may arise from the cellular processing thereof. In certain instances, the IL-13 sequences may comprise a signal peptide which may optionally be an exogenous, i.e. non-native, signal peptide. In other instances, the IL-13 proteins are mature proteins without a signal peptide.

The expression “IL-13R” (interleukin-13 receptor) as used herein typically refers to the “shared” IL-4/IL-13 receptor which consists of a complex formed by an IL-4Rα chain subunit and an IL-13Rα1 chain subunit, but may also include the “private” IL-13 receptor which consists of a single IL-13Rα2 chain subunit. The heterodimerization of IL-4Rα and IL-13Rα1 to form IL-13R is induced by IL-13 binding and promotes the activation of the Janus kinase (JAK)/Signal Transducer and Activator of Transcription (STAT) pathway, resulting in phosphorylation of STAT6. Phosphorylated STAT6 acts as a transcription factor activating many genes. IL-13 can also bind with very high affinity to the single chain IL-13Rα2, which is thought to function as a negative regulator of IL-13.

The IL-13R is typically mammalian (e.g. human, non-human primate (e.g. monkey) or mouse), preferably human. The term encompasses full-length, unprocessed subunits as well as any form of the subunit resulting from cellular processing. The term encompasses wild type proteins, naturally occurring variants, e.g. splice variants or allelic variants, as well as any other isoforms and mutant forms, as well as modified and unmodified forms of any of the foregoing. A reference to IL-13R includes proteins which may, for instance, be produced recombinantly or by synthetic methods and which have the same amino acid sequence as a naturally occurring or endogenous mammalian IL-13R subunit. Where the corresponding mammal is human, the protein may be referred to as hIL-13R. The nucleotide and amino acid sequences of IL-13R subunits from various species have been determined and are readily available from public sequence databases. The term hIL-13R encompasses a protein comprising or consisting of:
the exemplary IL-13Rα1 sequence accessible at UniProtKB Accession No P78552 or as set forth in SEQ ID NO: 5;
the exemplary IL-13Rα2 sequence accessible at UniProtKB Accession No. Q14627 or as set forth in SEQ ID NO. 6;
the exemplary IL-4Rα sequence accessible at UniProtKB Accession No. P24394 or as set forth in SEQ ID NO. 7;
   as well as biologically active fragments thereof and other sequences that may arise from the cellular processing thereof. In certain instances, the above sequences may comprise a signal peptide which may optionally be an exogenous, i.e. non-native, signal peptide. In other instances, the IL-13R proteins are mature proteins without a signal peptide.

There are different modes of binding able to antagonise IL-13 signalling. For example, lebrikizumab inhibits IL-13 signalling by binding to IL-13 with very high affinity and blocking IL-13 binding to IL-4Rα (Ultsch et al. J Mol Biol. 2013), while tralokinumab prevents IL-13 from binding to both IL-13Rα1 and IL-13Rα2 (Popovic et al. J Mol Biol. 2017). In both cases the phosphorylation of STAT6 and subsequent gene expression consequences are prevented

Assays to determine antagonism of IL-13 are known and any suitable assay may be used. Suitable assays include the use of cell lines which have been designed to assess the activation of the STAT6 pathway induced by IL-13. Suitable cell lines may express a reporter gene under the control of a STAT6 responsive promoter. For example, the cells may be modified to express secreted embryonic alkaline phosphatase (SEAP) under the control of the IFN-β minimal promoter fused to four STAT6 binding sites. In these cells, activation of the STAT6 pathway induces the expression of the reporter gene (e.g., SEAP). Expression of the reporter may be detected using any known method. For example, secretion of SEAP into the supernatant can readily be assessed using a SEAP detection reagent, such as QUANTI-Blue Solution. The cell line used in the assay must have a fully active STAT6 signalling pathway. Therefore, when the cell line is HEK (e.g., HEK293), the human STAT6 gene must be stably transfected. In one embodiment, the HEK cells used in the assay for IL-13 antagonism are HEK-Blue IL-4/IL-13 reporter cells (InvivoGen).

Alternatively, an IL-13-induced STAT6 phosphorylation assay in human primary keratinocytes (two donors) may be used to assay for IL-L3 antagonism. For instance, primary keratinocytes (e.g. NHEK, Adult skin, Lonza) may be cultured in keratinocyte serum-free medium (SFM) supplemented with bovine pituitary extract (25 μg/ml) and recombinant epidermal growth factor (EGF) (0.25 ng/ml). The keratinocytes are deprived of growth factors prior to stimulation and then stimulated with a serial dilution of human recombinant IL-13. After 10 to 60 stimulation, the intracellular levels of pSTAT6 are then determined, e.g., using AlphaLISA SureFire Ultra p-STAT6 (Tyr641) Assay Kit (ALSU-PST6-A500, PerkinElmer).

The expression “OX40L” (OX40 Ligand, Tumor necrosis factor ligand superfamily member 4) as used herein includes any native mammalian OX40L sequence (e.g. human, non-human primate (e.g. monkey) or mouse), preferably human OX40L. The term encompasses full-length, unprocessed OX40L as well as any form of OX40L resulting from cellular processing. The term encompasses wild type proteins, naturally occurring variants, e.g. splice variants or allelic variants, as well as any other isoforms and mutant forms, as well as modified and unmodified forms of any of the foregoing. A reference to OX40L includes proteins which may, for instance, be produced recombinantly or by synthetic methods and which have the same amino acid sequence as a naturally occurring or endogenous mammalian OX40L. Where the corresponding mammal is human, the protein may be referred to as hOX40L. The nucleotide and amino acid sequences of OX40L from various species have been determined and are readily available from public sequence databases. The term hOX40L encompasses the full-length, unprocessed 183 amino acid sequence of OX40L, such as that accessible at UniProtKB accession number P43489 or as set forth in SEQ ID No. 3, as well as biologically active fragments and other hOX40L sequences that may arise from the cellular processing thereof such as by proteases or alternative splicing (e.g. residues 51-183 of the full length hOX40L protein). An exemplary hOX40L sequence which comprises residues 51-183 of SEQ ID No. 3 is set forth in SEQ ID No. 4.

The expression “OX40” (OX40, Tumor necrosis factor receptor superfamily member 4) as used herein includes any native mammalian OX40 sequence (e.g. human, non-human primate (e.g. monkey) or mouse), preferably human OX40. The term encompasses full-length, unprocessed OX40 as well as any form of OX40 resulting from cellular processing. The term encompasses wild type proteins, naturally occurring variants, e.g. splice variants or allelic variants, as well as any other isoforms and mutant forms, as well as modified and unmodified forms of any of the foregoing. A reference to OX40 includes proteins which may, for instance, be produced recombinantly or by synthetic methods and which have the same amino acid sequence as a naturally occurring or endogenous mammalian OX40.

Where the corresponding mammal is human, the protein may be referred to as hOX40. The nucleotide and amino acid sequences of OX40 from various species have been determined and are readily available from public sequence databases. The term hOX40 encompasses a protein comprising or consisting of the exemplary OX40 sequence accessible at UniProtKB Accession No P43489 or as set forth in SEQ ID NO: 8, as well as biologically active fragments thereof and other sequences that may arise from the cellular processing thereof. In certain instances, the sequence may comprise a signal peptide which may optionally be an exogenous, i.e. non-native, signal peptide. In other instances, the OX40 protein is a mature proteins without a signal peptide.

OX40L is a member of the tumor necrosis factor superfamily that arranges forming a functional homotrimer. Three copies of OX40 bind to the trimeric ligand to form the OX40-OX40L complex. Receptor clustering is required for full activation of the signalling pathway. Antibodies binding to both OX40 and to OX40L have been described as able to antagonise intracellular signalling induced by OX40L-OX40 complex formation (Compaan et al. Structure 2006; Croft et al. Immunol Rev. 2009; Webb et al. Review. Clinic Rev Allerg Immunol. 2016; Guttman-Yassky et al. J Allergy Clin Immunol.

Assays to determine antagonism of OX40L are known and any suitable assay may be used. Suitable assays include those designed to detect bioactive OX40L by monitoring the activation of the NF-κB and AP-1 pathways. For example, the assays may use cell lines which express a reporter gene under the control of a NF-κB/AP-1-responsive promoter. According to these assays, binding of human OX40L to the homotrimeric OX40 receptor on the surface of these cells triggers a signalling cascade leading to the activation NF-κB and the subsequent production of the reporter gene.

Alternatively, OX40L-induced IL2 and IFNγ expression in CD3 positive T cells or PBMCs (from two donors) treated with suboptimal concentrations of anti-CD3 (primed) may be used to assay for OX40L-OX40 antagonism. For instance, PBMCs may be purified from fresh whole human blood from donors by density gradient centrifugation. The isolated PBMCs may then be plated in RPMI 1640 supplemented with 10% FBS, 2 mM L-glutamine, 100 U/mL penicillin, and 100 μg mL streptomycin and the cells then incubated with suboptimal concentrations of anti-CD3 and a serial dilution of human recombinant OX40L. The cells are incubated overnight and then IL2 and IFNy levels in the culture supernatant may be measured by ELISA (R&D Systems, #D2050 and #DY285) according to the manufacturer's instructions.

Alternatively, OX40L back-signalling in OX40L expressing cells can be used to assay for OX40L-OX40 antagonism. For instance, cells of the THP-1 cell line may be primed by LPS and incubated in the presence of OX40 (ECD) and/or benchmark and identified antibodies. IL6 levels may be measured by ELISA (R&D Systems, #D6050) according to the manufacturer's instructions. The internalization of OX40L may also be measured by FACS in the same conditions using serial dilutions of biotin-conjugated human OX40 protein or with biotin-conjugated anti-OX40L antibodies and stained with streptavidin-allophycocyanin for example.

The content of all database entries recited in the preceding paragraphs or elsewhere herein are hereby incorporated by reference in their entireties.

The terms IL-13, IL-13R, OX40L and OX40 as used herein typically refer to hIL-13, hIL-13R, hOX40L and hOX40, respectively. Accordingly, unless the context clearly indicates otherwise, references herein to IL-13, IL-13R, OX40L and OX40 are to be understood as being a reference to the humans version thereof.

### The Bispecific Antigen-Binding Molecules

As used herein the term “bispecific antigen-binding molecule” encompasses any antigen binding construct which has the ability to bind, and preferably neutralize biological function of, two different antigens/targets (in the invention (i) IL-13 or IL-13R and (ii) OX40L or OX40). A bispecific antigen-binding molecule can take a variety of forms and may for instance be a protein, polypeptide or molecular complex. A bispecific antigen-binding molecule may, for instance, be a single multifunctional polypeptide, or it may be a multimeric complex of two or more covalently or non-covalently associated polypeptides. In an exemplary embodiment the bispecific antigen-binding molecule is a bispecific antibody. The bispecific binding molecules of the present invention may advantageously display higher potency and/or efficacy in treating the disease or condition associated with or mediated by IL-13 and/or OX40L (e.g. AD) than treatment regimens which use a combination of mono-specific drugs each directed to one of the two targeted interactions.

The bispecific antigen-binding molecules of the present invention include anti-IL-13/OX40L binding molecules which specifically bind (i) IL-13 or IL-13R and (ii) OX40L or OX40. Methods for determining whether two molecules specifically bind one another are well known in the art. K_{D} may be used as the measure of the affinity for a binding molecule and its target/antigen. A lower K_{D} value is indicative of a higher affinity for a target. As used herein, the term “specifically binds” and the like may refer to where there is a binding affinity which is characterised by a K_{D} value of ≤1 μM, ≤500 nM, ≤250 nM, ≤100 nM ≤50 nM ≤25 nM ≤10 nM ≤5 nM ≤2.5 nM ≤2 nM, ≤1 nM, ≤0.5 nM, ≤0.4 nM , ≤0.25 nM, ≤0.1 nM, ≤0.05 nM, ≤0.01 nM, ≤0.005 nM, or ≤0.001 nM.

Accordingly, an anti-IL-13/OX40L bispecific antigen-binding molecule of the invention may specifically bind IL-13 or IL-13R with a K_{D} value of ≤1 μM, ≤500 nM, ≤250 nM, ≤100 nM ≤50 nM ≤25 nM ≤10 nM ≤5 nM ≤2.5 nM ≤2 nM, ≤1 nM, ≤0.5 nM, ≤0.4 nM , ≤0.25 nM, ≤0.1 nM, ≤0.05 nM, ≤0.01 nM, ≤0.005 nM, or ≤0.001 nM and/or may specifically bind OX40L or OX40 with a K_{D} value of ≤1 μM, ≤500 nM, ≤250 nM, ≤100 nM ≤50 nM ≤25 nM ≤10 nM ≤5 nM ≤2.5 nM ≤2 nM, ≤1 nM, ≤0.5 nM, ≤0.4 nM , ≤0.25 nM, ≤0.1 nM, ≤0.05 nM, ≤0.01 nM, ≤0.005 nM, or ≤0.001 nM.

Various methods exist in the method art for determining the value of the dissociation constant K_{D}, such as surface plasmon resonance (SPR). In the context of the present invention, the K_{D} value is preferably determined using Surface Plasmon Resonance at 25° C. and/or 37° C. e.g. with a Biacore™ system.

In certain embodiments, an anti-IL-13/IL-13R & OX40L/OX40 bispecific antigen-binding molecule may comprise an anti-IL-13 or anti-IL13R antibody (e.g. a human or humanised anti-IL-13 or anti-IL-13R antibody) or antigen binding fragment thereof and/or may comprise an anti-OX40L or anti-OX40 antibody (e.g. a human or humanised anti-OX40L or anti-OX40 antibody) or antigen binding fragment thereof. In some embodiments, the bispecific antigen-binding molecule is a bispecific antibody, e.g. a bivalent, trivalent or tetravalent bispecific antibody. In certain embodiments the bispecific antigen-binding molecule is an antibody in the DVD-Ig format and/or is a polypeptide complex comprising variable domains of an antibody and T cell receptor (TCR) constant regions, wherein the TCR constant regions are capable of forming a dimer comprising at least one non-native interchain bond (a complex of this type is described WO2019057122 and may be referred to herein as a WuXibody).

### Antigen Binding Domains and Other Components

Bispecific antigen-binding molecules of the present invention include bispecific antigen-binding molecules which comprise a first antigen binding domain (B1) which is an IL-13 or IL-13R antigen binding domain and a second antigen binding domain (B2) which is an OX40L or OX40 antigen binding domain; such a bispecific antigen-binding molecule may be referred to generally herein as “an anti-IL-13/IL-13R & OX40L/OX40 bispecific antigen-binding molecule”. Within this general class of molecule of the invention, the following specific embodiments are provided:
(i) a bispecific antigen-binding molecule which specifically binds to IL-13 and OX40L (optionally referred to herein as “an anti-IL-13/OX40L bispecific antigen-binding molecule”);
(ii) a bispecific antigen-binding molecule which specifically binds to IL-13 and OX40 (optionally referred to herein as “an anti-IL-13/OX40 bispecific antigen-binding molecule”);
(iii) a bispecific antigen-binding molecule which specifically binds to IL-13R and OX40L (optionally referred to herein as “an anti-IL-13R/OX40L bispecific antigen-binding molecule”); and
(iv) a bispecific antigen-binding molecule which specifically binds to IL-13R and OX40 (optionally referred to herein as “an anti-IL-13R/OX40 bispecific antigen-binding molecule”).

In other words, the bispecific antigen-binding molecule is a molecule wherein:
i) B1 specifically binds to IL-13 and B2 specifically binds to OX40L;
ii) B1 specifically binds to IL-13R and B2 specifically binds to OX40L;
iii) B1 specifically binds to IL-13 and B2 specifically binds to OX40; or
iv) B1 specifically binds to IL-13R and B2 specifically binds to OX40.
   Option (i) is particularly preferred.

The anti-IL-13/IL-13R & OX40L/OX40 bispecific antigen-binding molecules of the invention may comprise one or more further IL-13 or IL-13R binding domains. Thus, embodiments are envisaged where there are e.g. two or three IL-13 binding domains, or e.g. two or three IL-13R binding domains. In embodiments where there is more than one IL-13 binding domain, the two or more of the IL-13 binding domains may be identical, substantially identical or different to one another. In embodiments where there is more than one IL-13R binding domain, the two or more of the IL-13R binding domains may be identical, substantially identical or different to one another.

The anti-IL-13/IL-13R & OX40L/OX40 bispecific antigen-binding molecules of the invention may comprise one or more further OX40L or OX40 binding domains. Thus, embodiments are envisaged where there are e.g. two or three OX40L binding domains, or e.g. two or three OX40 binding domains. In embodiments where there is more than one OX40L binding domain, the two or more of the OX40L binding domains may be identical, substantially identical or different to one another. In embodiments where there is more than one OX40 binding domain, the two or more of the OX40 binding domains may be identical, substantially identical or different to one another.

The terms “antigen-binding domain” and “binding domain” and the like may be used interchangeably herein. An antigen-binding domain is typically capable of specifically binding a particular antigen of interest (e.g. IL-13, IL-13R, OX40L, or OX40), such as specifically binding with a K_{D} value of ≤1 μM, ≤500 nM, ≤250 nM, ≤100 nM, ≤50 nM, ≤25 nM, ≤10 nM, ≤5 nM, ≤2.5 nM, ≤2 nM, ≤1 nM, ≤0.5 nM, ≤0.4 nM , ≤0.25 nM, ≤0.1 nM, ≤0.05 nM, ≤0.01 nM, ≤0.005 nM, or ≤0.001 nM.

Examples of antigen-binding domains that may be used in the present invention include immunoglobulin-based antigen-binding domains and non-immunoglobulin-based antigen-binding domains. Thus, examples of antigen-binding domains include binding domains derived from an immunoglobulin or antibody or from a source other than an immunoglobulin or antibody (e.g. from a proteinaceous binding molecule with immunoglobulin-like binding properties). As used herein, the term “derived from” and the like includes a reference to where a given entity (e.g. an antigen-binding domain, an antibody) may be obtained from a particular source, whether directly or indirectly, and optionally with one or more modifications such as with one or more mutations.

An antigen-binding domain (e.g. an IL-13 or IL-13R antigen-binding domain or an OX40L or OX40 antigen-binding domain) may for example comprise or consist of an antibody or an antigen-binding fragment thereof, e.g. Fabs, scFabs, Fvs, and scFvs. Non-limiting examples of antigen-binding fragments which may be used in the practice of the present invention include Fab fragments, F(ab′)₂ fragments, Fab′ fragments, Fd fragments, Fv fragments, dAb fragments, isolated complementarity determining region (CDR)s, single chain antibodies such as scFv and heavy chain antibodies such as VHH and camel antibodies as well as other antigen-binding fragments disclosed elsewhere herein. Typically an antigen-binding fragment of an antibody comprises one or more CDRs (e.g. HCDR3 optionally in combination with one of more further CDRs (e.g. a set of six CDRs from an HCVR/LCVR pair)).

Antibodies for use in the present invention may be obtained by any suitable means. For instance, antibodies may be obtained by administering polypeptides to an animal, e.g. a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable. Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp77-96, Alan R Liss, Inc., 1985). Antibodies may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15): 7843-78481; WO 92/02551; WO 2004/051268 and WO 2004/106377. Antibodies can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Methods for obtaining and identifying antibodies which may be used in the practice of the present invention also include those described in the ImmunoQure patent applications WO2013/098419 (“Methods of Providing Monoclonal Auto-Antibodies with Desired Specificity”), WO2013/098420 (“Method of Isolating Human Antibodies”), and WO2015/001407 (“Method of Providing Anti-Human Cytokine Antibodies for Pharmaceutical Use”).

Non-antibody antigen-binding domains are also contemplated for use in the practice of the present invention. Accordingly, an antigen-binding domain (e.g. an IL-13 or IL-13R antigen-binding domain or an OX40L or OX40 antigen binding domain) may, for example, be derived from, or comprise or consist of, a non-antibody scaffold protein, a DARPin (designed ankyrin repeat proteins), an anticalin or an lipocalin, an affibody, an avimer, an adnectin, an atrimer, or an evasin etc.

Combinations of different types of antigen-binding domains described herein are contemplated within a bispecific antigen-binding molecule of the invention. Thus, for instance, the antigen-binding domains can each independently comprise or consist of an antibody or an antigen-binding fragment of an antibody or be derived from, or comprise or consist of, a non-antibody scaffold protein, a DARPin (designed ankyrin repeat proteins), an anticalin or an lipocalin, an affibody, an avimer, an adnectin, an atrimer, or an evasin etc.

In certain embodiments, B1 and/or B2 may comprise or consist of an antibody (e.g. an IgG antibody such as IgG1 or IgG4).

In certain embodiments, B1 and/or B2 may comprise or consist of an antigen-binding fragment of an antibody (e.g. an Fv fragment (e.g. scFv), a Fab fragment).

In certain embodiments, B1 and/or B2 may be derived from, or comprise or consist of, a non-antibody scaffold protein, a DARPin (designed ankyrin repeat proteins), an anticalin or an lipocalin, an affibody, an avimer, an adnectin, an atrimer, or an evasin etc.

In certain embodiments, B1 comprises or consists of an antibody (e.g. an IgG antibody such as IgG1 or IgG4) and B2 comprises or consists of an antigen-binding fragment of an antibody (e.g. an Fv fragment (e.g. scFv), a Fab fragment).

In certain embodiments, B1 comprises or consists of an antigen-binding fragment of an antibody (e.g. an Fv fragment (e.g. scFv), a Fab fragment) and B2 comprises or consists of an antibody (e.g. an IgG antibody such as IgG1 or IgG4).

A bispecific antigen-binding molecule of the invention may be produced by any suitable means. For example, all or part of the molecule may be expressed as a fusion protein by a cell comprising a nucleotide which encodes said molecule. Alternatively parts of the molecule may be produced separately, for example by expression from separate nucleotides optionally in separate cells, and then subsequently joined together.

In addition to the at least two antigen binding domains, the bispecific antigen-binding molecules may optionally comprise one or more further components. Such one or more further components may, for instance, facilitate the association or binding of the antigen binding domains with each other. Non-limiting examples of one or more further components which may be incorporated into a bispecific antigen-binding molecule of the invention include linkers (e.g. peptide linkers and hinge regions) and Fc domains as well as fragments thereof such as a heavy chain Fc region or a CH3 domain. Thus, in certain embodiments a bispecific antigen-binding molecule of the invention comprises an Fc domain, preferably a human Fc domain, or a fragment thereof. A human Fc domain may be a native or variant human Fc domain.

An Fc domain is composed of two polypeptide chains, each referred to as a heavy chain Fc region, which dimerize to form the Fc domain. An Fc domain may be a native or variant Fc domain (e.g. with one or more amino acid insertions, deletions or substitutions). An Fc domain may for instance be modified or engineered to render it better suited for its intended pharmacological use, e.g. to alter (e.g. increase) half-life and/or to alter effector function. Preferably an Fc domain is a human Fc domain. An Fc domain or region may be from any suitable class of an antibody, e.g. IgA, IgD, IgE, IgG, or IgM, or subclass thereof (e.g. IgA1, IgA2, IgG1, IgG2, IgG3 or IgG4). Preferably, the Fc domain is human and/or is an IgG domain, e.g. IgG1 or IgG4. In a native antibody the Fc regions within an Fc domain are typically identical, but for the purpose of the present invention the two Fc regions within an Fc domain, if present, may be the same or different e.g. from different antibody classes, or subclasses (e.g. from two different IgG classes).

The term “linker” and the like as used herein includes a reference to any molecule or entity that joins two or more different components of a bi specific antigen-binding molecule of the invention. Examples of linkers include peptide linkers, and non-immunoglobulin polypeptides such as albumin (e.g. two or more antigen-binding domains may be linked to albumin (e.g. HSA) to form an bispecific antigen-binding molecule comprising the two or more antigen binding domains each bound to an albumin molecule). A hinge region may also be used to link components of the antigen-binding molecules of the invention e.g. to bind an antigen-binding domain (e.g. in the form of an antigen-binding fragment of an antibody such as a Fab fragment) to an Fc region. Hinge regions are typically found at the N-termini of Fc regions. A hinge region may be a native or modified /variant hinge region.

Components of the antigen-binding molecules of the invention (e.g. B1 and B2) may be connected be to one another by any suitable means. Components may be directly connected to one another, or indirectly connected to one another by one or more suitable molecules (e.g. by a linker or hinge region). Thus, for example, a bispecific antigen-binding molecule of the invention may comprise or consist of a fusion protein comprising B1 and B2, optionally joined by a peptide linker. Various combinations of direct and/or indirect means are envisaged within the practice of the present invention, and thus a variety of direct and/or indirect means may be employed to connect the components of a bispecific antigen-binding molecule of the invention. In some embodiments, the antigen binding domains are connected to one another through an Fc domain or a fragment thereof. Typically, Fc domains require the use of hinge regions and therefore the antigen binding domains may, for example, be connected through an Fc domain via one or more hinge regions.

A bispecific antigen-binding molecule of the invention may optionally be linked directly or indirectly to a further moiety e.g. a therapeutic moiety. Thus, in some embodiments the bispecific antigen-binding molecule (e.g. bispecific antibody) is conjugated to one or more additional therapeutic agents.

### Bispecific Antibodies

The bispecific antigen-binding molecule of the invention may be a bispecific antibody. Antibodies of the invention are typically monoclonal antibodies. An antibody of the invention may for instance be a chimeric antibody, a CDR-grafted antibody, a nanobody, a human or humanised antibody or an antigen-binding fragment of any of the foregoing. Typically, the antibody is a human antibody. As discussed above, an antibody may comprise a complete antibody molecule having full length heavy and light chains or an antigen-binding fragment thereof. Accordingly, an antibody of the invention may comprise or consist of a complete antibody molecule having full length heavy and light chains, or it may comprise or consist of an antigen-binding fragment thereof.

The constant region domains of the bispecific antibody molecule of the present invention, if present, may be selected having regard to the proposed function of the antibody molecule, and in particular the effector functions which may be required. For example, the constant region domains may be human IgA, IgD, IgE, IgG or IgM domains. Typically, the constant region domains are human. In particular, human IgG (i.e. IgG1, IgG2, IgG3 or IgG4) constant region domains may be used, e.g. a human IgG1 or IgG4 constant region domain. The light chain constant region may be either lambda or kappa.

The bispecific antibody of the invention may be a human antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. A human antibody may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

Fully human antibodies are those antibodies in which the variable regions and the constant regions (where present) of both the heavy and the light chains are all of human origin, or substantially identical to sequences of human origin, but not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP 0546073, U.S. Pat. Nos. 5,545,806, 5,569,825, 5,625,126, 5,633,425, 5,661,016, 5,770,429, EP 0438474 and EP 0463151.

The term “humanized antibody” is intended to refer to CDR-grafted antibody molecules in which CDR sequences derived from the germline of another mammalian species, such as a mouse, have been grafted onto human framework sequences. Additional framework region modifications may be made within the human framework sequences. As used herein, the term ‘CDR-grafted antibody molecule’ refers to an antibody molecule wherein the heavy and/or light chain contains one or more CDRs (including, if desired, one or more modified CDRs) from a donor antibody (e.g. a murine or rat monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs are transferred to the human antibody framework (see for example, Kashmiri et al., 2005, Methods, 36, 25-34). When the CDRs or specificity determining residues are grafted, any appropriate acceptor variable region framework sequence may be used having regard to the class/type of the donor antibody from which the CDRs are derived, including mouse, primate and human framework regions. Suitably, the CDR-grafted antibody according to the present invention has a variable domain comprising human acceptor framework regions as well as one or more of the CDRs or specificity determining residues.

Antibodies of the invention may be “isolated” antibodies. An isolated antibody is an antibody which is substantially free of other antibodies having different antigenic specificities.

A wide variety of bispecific antibody formats and production methods are known in the art and any suitable format and production method may be used in the practice of the present invention. There are many available bispecific antibody formats but, generally speaking, bispecific antibodies can be divided into IgG-like and non-IgG like bispecific antibodies. IgG-like bispecific antibodies comprise an Fc domain and may be further categorized into symmetric IgG-like bispecific antibodies (e.g. dual variable domain (DVD)-Igs) and non-symmetric IgG-like bispecific antibodies. Non-IgG-like bispecific antibodies lack an Fc domain and may for instance be made by fusing two different antigen-binding antibody fragments to a non-immunoglobulin protein (e.g. human serum albumin (HSA)), by directly fusing two antigen-binding antibody fragments, or by chemical conjugation of two different antibodies or smaller antigen-binding antibody fragments. Any of these an IgG-like and non-IgG-like formats and production techniques may be employed in the practice of the present invention. For a review of bispecific antibodies including exemplary bispecific antibody formats and production methods which may be used in the practice of the present invention (such as those outlined in the discussion below), reference is made to Kontermann and Brinkmann (2017), “The making of bispecific antibodies”, Mabs, February-March 9(2): 182-199; Kontermann and Brinkmann (July 2015), “Bispecific antibodies”, Drug Discovery Today, 20(7): 838-847; Sedykh et al. (2018), “Bispecific antibodies: design, therapy, perspectives”, Drug Design, Development and Therapy, 12: 195-208; and Fan et al. (2015), “Bispecific antibodies and their applications”, Journal of Hematology and Oncology, 8:130.

Exemplary bispecific antibodies and techniques according to the present invention include but are not limited to asymmetric IgG-like, symmetric IgG-like (e.g. comprising two Fab regions and an Fc domain), non-IgG-like, quadromas, WuXibodies, DVD-Igs, knob-in-hole (kih), IgG-scFv fusions, two-in-one or dual action Fab (DAF) antibodies, half molecule exchange, κλ-bodies, CrossMab, CrossFab, Triomab, (SEED)body, leucine zipper, common light chain (e.g. kih IgG common LC), ortho-Fab IgG, 2 in 1-IgG, scFv2-Fc, triabodies, scFv-based or diabody bispecific formats, tandem scFvs, single-chain diabodies, nanobodies, dock-and-lock (DNL) method, bi-Nanobody, bispecific T-cell engagers (BiTEs), tandem diabodies (tandAbs), chemically linked Fabs, bivalent and trivalent scFvs, Dual affinity retargeting (DARTs), DART-Fc, scFv-HSA-scFv, and DNL-Fab3 bispecific antibodies.

In one embodiment, the bispecific antibody is a dual-variable domain immunoglobulin (DVD-Ig). A DVD-Ig can be generated from two parental mAbs by placing two variable domains from one of the parental mAbs onto the heavy chain and the light chain of the other parental antibody, instead of one variable domain, to yield a tetravalent IgG-like molecule.

A bispecific antibody of the present invention may be a WuXibody. A detailed description of WuXibodies may be found in WO 2019/057122 (WuXi Biologics). The term “WuXiBody” includes bispecific antibodies comprising soluble chimeric protein with variable domains of an antibody and T cell receptor (TCR) constant regions, wherein the TCR constant regions are capable of forming a dimer comprising at least one non-native interchain bond; such WuXibodies are described in more detail in WO 2019/057122 along with methods for their production and various possible WuXibody formats.

More specifically, the term WuXibody as used herein may refer to a bispecific polypeptide complex, comprising a first antigen-binding moiety associated with a second antigen-binding moiety, wherein:
(i) the first antigen-binding moiety comprises: a first polypeptide comprising, from N-terminus to C-terminus, a first heavy chain variable domain (VH) of a first antibody operably linked to a first TCR constant region (C1) , and a second polypeptide comprising, from N-terminus to C-terminus, a first light chain variable domain (VL) of the first antibody operably linked to a second TCR constant region (C2) , wherein: C1 and C2 are capable of forming a dimer comprising at least one non-native interchain bond between a first mutated residue comprised in C1 and a second mutated residue comprised in C2, and the non-native interchain bond is capable of stabilizing the dimer, and the first antibody has a first antigenic specificity;
(ii) the second antigen-binding moiety has a second antigenic specificity which is different from the first antigenic specificity,
   and the first antigen-binding moiety and the second antigen-binding moiety are less prone to mispair than otherwise would have been if both the first and the second antigen-binding moieties are counterparts of natural Fab.

In the bispecific antibodies of the present invention the first antigenic specificity of the WuXibody may be directed to IL-13 or IL-13R and the second antigenic specificity may be directed to OX40L or OX40. Alternatively, the first antigenic specificity may be directed to OX40L or OX40 and the second antigenic specificity may be directed to IL-13 or IL-13R.

Examples of pairs of TCR constant regions (C1 and C2) which may be used in the WuXibodies of the present invention include, for example, alpha/beta, pre-alpha/beta, and gamma/delta TCR constant regions. The TCR constant regions can be in full length or in a fragment, and can be engineered (e.g. to comprise one or more mutations), as long as the pair of TCR constant regions are capable of associating with each other to form a dimer.

The WuXiBodies of the present invention may be provided in any suitable bispecific format. Examples of bispecific formats include those described herein as well as the bispecific formats described in WO2019/057122. Antigen-binding fragments of WuXiBodies are included within the scope of the present invention and are also described in WO2019/057122.

### Pharmaceutical Compositions

A bispecific antigen-binding molecule of the invention (e.g. a bispecific antibody of the invention) may be formulated for administration as a pharmaceutical composition. Accordingly a bispecific antigen-binding molecule of the invention may be provided in the form of a pharmaceutical composition comprising the bispecific antigen-binding molecule and a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion), topical or oral administration. Preferred pharmaceutically acceptable carriers comprise aqueous carriers or diluents. Examples of suitable aqueous carriers that may be employed in the compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be preferable to include isotonic agents, for example, sugars, polyalcohols such as mannitol, sorbitol, or sodium chloride in the composition.

Pharmaceutical compositions typically must be sterile and stable under the conditions of manufacture and storage. The composition can be formulated as a solution, microemulsion, liposome, or other ordered structure suitable to high drug concentration.

The pharmaceutical compositions of the invention may comprise one or more additional active ingredients as well as a bispecific antigen-binding molecule of the invention.

### Therapeutic Uses

In a further aspect of the invention, there is a provided a bispecific antigen-binding molecule of the invention for use as a medicament. Methods of treatment of a disease or condition are also provided and comprise administering a bispecific antigen-binding molecule of the invention to a subject in need thereof to thereby treat the disease or condition.

As discussed above, IL-13 and OX40L have been identified as important factors in a variety of diseases and conditions and, as such, the bispecific antigen-binding molecules of the invention may be used to treat such diseases and conditions. Accordingly, one aspect of the present invention provides a method of treating a disease or condition which is associated with or mediated by IL-13 and/or OX40L in a patient, the method comprising administering to the patient an anti-IL-13/IL-13R & OX40L/OX40 bispecific antigen-binding molecule (e.g. a bispecific antibody) of the invention. The invention also provides an anti-IL-13/IL-13R & OX40L/OX40 bispecific antigen-binding molecule of the invention for use in a method of treating a disease or condition which is associated with or mediated by IL-13 and/or OX40L. The invention also provides an anti-IL-13/IL-13R & OX40L/OX40 bispecific antigen-binding molecule of the invention for use in the manufacture of a medicament for the treatment of a disease or condition which is associated with or mediated by IL-13 and/or OX40L.

The phrase “a disease or condition which is associated with or mediated by” and the like in relation to a particular cytokine (here IL-13 or OX40L) includes a reference to a disease or condition which is associated with or mediated by expression, signalling or activity of the cytokine, or treatable by antagonism of the cytokine e.g. by blocking the interaction between the cytokine and a ligand for the cytokine or by otherwise inhibiting the activity and/or signalling of the cytokine. Examples of diseases or conditions which are associated with or mediated by IL-13 and/or OX40L include dermatological diseases (e.g., atopic dermatitis, prurigo nodularis, chronic hand eczema, allergic dermatitis, psoriasis, lichen planus, hidradenitis suppurativa), asthma, allergic diseases (e.g., allergic rhinitis), cardiovascular diseases (e.g., myocardial infarction, cardiac hypertrophy-related diseases), atherosclerosis, musculoskeletal diseases (rheumatoid arthritis), COPD, age-related macular degeneration, periodontitis uveitis, cancer, inflammatory bowel disease, fibrosis, scleroderma, or eosinophilic esophagitis.

The bispecific antigen-binding molecules of the invention may be useful in treating a dermatological disease or condition e.g. atopic dermatitis, prurigo nodularis, chronic hand eczema, allergic dermatitis, psoriasis, lichen planus or hidradenitis suppurativa. Accordingly, the invention provides a method of treating a dermatological disease or condition in a patient comprising administering to the patient a bispecific antigen-binding molecule (e.g. a bispecific antibody) of the invention. The invention also provides a bispecific antigen-binding molecule of the invention for use in a method of treating a dermatological disease or condition. The invention also provides a bispecific antigen-binding molecule of the invention for use in the manufacture of a medicament for the treatment of a dermatological disease or condition.

In the present invention, treatment may be in respect of a patient with the disease or condition, or may be in respect of a patient in whom the disorder is to be prevented such as patient which is prone to, or at risk of, having the disease or condition. Thus, the term “treatment” and the like as used herein encompasses therapeutic and prophylactic treatment. The term “treatment” may for instance refer to preventing the disease or condition from occurring, delaying the onset of the disease or one or more symptoms thereof, causing regression of the disease or medical condition in a patient, suppressing the disease or medical condition (e.g. slowing the development of the disease or medical condition, or reducing the severity and/or frequency of flares), or alleviating to some extent one or more of the symptoms of the disease or medical condition in a patient. In the case of AD, symptoms include pruritus, erythema, edema, xerosis, erosions/excoriations, oozing and crusting, lichenification, impaired skin barrier, and redness. The term “treatment” and the like does not necessarily entail complete treatment or prevention and the term may therefore encompass varying degrees of treatment or prevention.

In therapeutic applications, administration is to a subject already suffering from the disease or condition. Such therapeutic treatment may for instance cure, alleviate or partially arrest the disease or condition or one or more of its symptoms. Accordingly, therapeutic administration may result in a decrease in symptom severity, or an increase in frequency or duration of symptom-free periods. An amount adequate to accomplish a therapeutically useful effect may be referred to as a “therapeutically effective amount”.

In prophylactic applications, administration is to a subject not yet, or not currently, exhibiting symptoms of the disease or condition. Such prophylactic treatment may for instance prevent, delay, or reduce in severity the development of the disease or condition or one or more of its symptoms. An amount adequate to accomplish a prophylactically useful effect may be referred to as a “ prophylactically effective amount”. The subject may have been identified as being at risk of developing the disease or condition by any suitable means. The patient may be prone to, or at risk of, having the disease or condition (e.g. a patient with a family or individual history of the disease or condition) or in whom the disorder is to be prevented.

Therapeutically and prophylactically effective amounts will depend on the severity of the disease or condition as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” as used herein may refer to an improvement in the severity of the disease or condition or quality of life (QoL). Various test procedures and scoring systems are available for assessing disease severity (e.g. mild, moderate, moderate-to-severe, or severe) and quality of life and any one or more suitable measures may be used. An overview of disease severity and quality of life measures for AD may be found in e.g. Rehal and Armstrong (2011), Plos ONE 6(4):e17520, and in Gooderham et al. (2018), J Cutan Med Surg., 22(IS) 10S-16S). One common measure of disease severity in AD patients is the Eczema Area Severity Index (EAST). Other examples of suitable disease severity and QoL measures for AD include: SCORing Atopic Dermatitis (SCORAD), the Body Surface Area (BSA) assessment, the Physician's Global Assessments (PGA), Investigator Global Assessment (IGA), Dermatitis Severity Index (ADSI), Six Area, Six Sign Atopic Dermatitis (SASSAD), Investigators' Global Atopic Dermatitis Assessment (IGADA), the Pruritus—Visual Analogue Scale (Pruritus-VAS), 5-D Itch (Pruritis) Scale, Dermatology Life Quality Index (DLQI), Children's Dermatology Life Quality Index (CDLQI), Dermatitis Family Impact (DFI), and Infant's Dermatology, and Quality of Life (IDQOL), and the Medical Outcome Sleep Study (MOSS).

A bispecific antigen binding molecule of the invention may, for example, be used to treat acute or chronic AD. A bispecific antigen binding molecule of the invention may be used to treat mild, moderate, moderate-to-severe, or severe AD. Disease severity can easily be determined by a skilled person using standard test procedures such as by using one or more of the above-mentioned measures of disease severity or quality of life measures for AD, e.g. EASI.

The terms “patient” and “subject” are used interchangeably herein and the terms include a reference to any human or non-human animal (preferably a mammal). The term “mammal” as used herein refers to any member of the class Mammalia, including, without limitation, humans and non-human primates such as chimpanzees and other apes and monkey species; farm animals such as cattle, sheep, pigs, goats and horses; domestic/companion animals such as dogs and cats; as well as rabbits and rodents such as mice, rats, and guinea pigs, and the like. Typically, the invention relates to administration to a human patient. The human patient may be an adult patient (18 years or older). Alternatively the human patient may be a paediatric patient (less than 18 years old). In some instances, the patient may be less than 12 years old. In certain embodiments, the subject is a human patient who has been identified as having a disorder or condition likely to respond to a bispecific antigen-binding molecule of the invention.

### Administration

The bispecific antigen-binding molecules and pharmaceutical compositions of the present invention may be administered via one or more routes of administration using one or more of a variety of methods known in the art. As will be appreciated by the skilled artisan, the route and/or mode of administration will vary depending upon the desired results. Routes of administration may include intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection. In some embodiments a bispecific antigen-binding molecule of the invention (e.g. a bispecific antibody of the invention) is administered to the patient by injection, preferably by subcutaneous or intravenous injection. Alternatively, a bispecific antigen-binding molecule of the invention can be administered via a non-parenteral route, such as by topical or oral administration.

A suitable dosage of a bispecific antigen-binding molecule of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular bispecific antigen-binding molecule employed, the route of administration, the time of administration, the rate of excretion of the bispecific antigen-binding molecule, the duration of the treatment, other drugs, compounds and/or materials used in combination with the particular compositions employed, the age, sex, weight, condition, general health and prior medical history of the patient being treated, and like factors well known in the medical arts.

A suitable dose of a bispecific antigen-binding molecule of the invention may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

The initial dose may be followed by administration of a second or plurality of subsequent doses. The second and subsequent doses may be separated by an appropriate time. Dosage and frequency may vary depending on the half-life of the bispecific antigen-binding molecule in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms.

### Additional Medications/Separate Treatment Methods

A bispecific antigen-binding molecule of the invention may be administered in combination with an additional medication and/or treatment method for the prevention or treatment of the disease or condition. Combined administration of two or more agents may be achieved in a number of different ways. In one embodiment, the bispecific antigen-binding molecule and the other agent may be administered together in a single composition. In another embodiment, the bispecific antigen-binding molecule and the other agent may be administered in separate compositions as part of a combined therapy. For example, the bispecific antigen-binding molecule may be administered prior to, concurrent with, or after the other agent. The separate compositions may be administered by the same route, or by different routes.

Examples of suitable medications and/or treatments are described in the art. For instance, in relation to AD see e.g. Dhadwal et al. (2018), J Cutan Med Surg., 22(IS) 21S-29S). In the case of AD examples include topical treatments (such as topical corticosteroids, and topical calcineurin inhibitors), phototherapy, and systemic treatments (such as systemic corticosteroids, methotrexate, cyclosporine A, mycophenolate, and azathioprine).

### Brief Description of the Sequence Listing

SEQ ID NO: 1 is the amino acid sequence of an exemplary human IL-13 sequence including the signal peptide (corresponding to Swiss-Prot Accession No. P35225).

SEQ ID NO: 2 is the amino acid sequence of an exemplary human IL-13 sequence without the signal peptide of amino acid residues 1 to 35 of SEQ ID NO.1.

SEQ ID NO: 3 is the amino acid sequence of an exemplary full length unprocessed human OX40L sequence (corresponding to UniProtKB accession number P43489).

SEQ ID NO: 4 is the amino acid sequence of an exemplary human OX40L sequence corresponding to residues 51-183 of SEQ ID NO.3.

SEQ ID NO: 5 is the amino acid sequence of an exemplary human IL-13R alpha 1 sequence (corresponding to UniProtKB accession number P78552).

SEQ ID NO: 6 is the amino acid sequence of an exemplary human IL-13R alpha 2 sequence (corresponding to UniProtKB accession number Q14627).

SEQ ID NO: 7 is the amino acid sequence of an exemplary human IL-4R alpha sequence (corresponding to UniProtKB accession number P24394).

SEQ ID NO: 8 is the amino acid sequence of an exemplary human OX40 sequence (corresponding to UniProtKB accession number P43489).

Informal Sequence Listing

```
SEQ ID NO: 1
                10 20 30 40
MHPLLNPLLL ALGLMALLLT TVIALTCLGG FASPGPVPPS
                50 60 70 80
TALRELIEEL VNITQNQKAP LCNGSMVWSI NLTAGMYCAA
                90 100 110 120
LESLINVSGC SAIEKTQRML SGFCPHKVSA GQFSSLHVRD
              130 140
TKIEVAQFVK DLLLHLKKLF REGRFN
SEQ ID NO: 2
PVPPSTALRELIEELVNITQNQKAPLCNGSMVWSINLTAGMYCAALESL
INVSGCSAIEKTQRMLSGFCPHKVSAGQFSSLHVRDTKIEVAQFVDKDL
LLHLKKLFREGRFN
SEQ ID NO: 3
MERVQPLEENVGNAARPRFERNKLLLVASVIQGLGLLLCFTYICLHFSA
LQVSHRYPRIQSIKVQFTEYKKEKGFILTSQKEDEIMKVQNNSVIINCD
GFYLISLKGYFSQEVNISLHYQKDEEPLFQLKKVRSVNSLMVASLTYKD
KVYLNVTTDNTSLDDFHVNGGELILIHQNPGEFCVL
SEQ ID NO: 4
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL
SEQ ID NO: 5
MEWPARLCGLWALLLCAGGGGGGGGAAPTETQPPVTNLSVSVENLCTVI
WTWNPPEGASSNCSLWYFSHFGDKQDKKIAPETRRSIEVPLNERICLQV
GSQCSTNESEKPSILVEKCISPPEGDPESAVTELQCIWHNLSYMKCSWL
LPGRNTSPDTNYTLYYWHRSLEKIHQCENIFREGQYFGCSFDLTKVKDS
SFEQHSVQIMVKDNAGKIKPSFNIVPLTSRVKPDPPHIKNLSFHNDDLY
VQWENPQNFISRCLFYEVEVNNSQTETHNVFYVQEAKCENPEFERNVEN
TSCFMVPGVLPDTLNTVRIRVKTNKLCYEDDKLWSNWSQEMSIGKKRNS
TLYITMLLIVPVIVAGAIIVLLLYLKRLKIIIFPPIPDPGKIFKEMFGD
QNDDTLHWKKYDIYEKQTKEETDSVVLIENLKKASQ
SEQ ID NO: 6
MAFVCLAIGCLYTFLISTTFGCTSSSDTEIKVNPPQDFEIVDPGYLGYL
YLQWQPPLSLDNFKECTVEYELKYRNIGSETWKTIITKNLHYKDGFDLN
KGIEAKIHTLLPWQCTNGSEVQSSWAETTYWISPQGIPETKVQDMDCVY
YNWQYLLCSWKPGIGVLLDTNYNLFYWYEGLDHALQCVDYIKADGQNIG
CRFPYLEASDYKDFYICVNGSSENKPIRSSYFTFQLQNIVKPLPPVYLT
FTRESSCEIKLKWISPLGPIPARCFDYEIEIREDDTTLVTATVENETYT
LKTTNETRQLCFVVRSKVNIYCSDDGIWSEWSDKQCWEGEDLSKKTLLR
FWLPFGFILILVIFVTGLLLRKPNTYPKMIPEFFCDT
SEQ ID NO: 7
MGWLCSGLLFPVSCLVLLQVASSGNMKVLQEPTCVSDYMSISTCEWKMN
GPTNCSTELRLLYQLVFLLSEAHTCIPENNGGAGCVCHLLMDDVVSADN
YTLDLWAGQQLLWKGSFKPSEHVKPRAPGNLTVHTNVSDTLLLTWSNPY
PPDNYLYNHLTYAVNIWSENDPADFRIYNVTYLEPSLRIAASTLKSGIS
YRARVRAWAQCYNTTWSEWSPSTKWHNSYREPFEQHLLLGVSVSCIVIL
AVCLLCYVSITKIKKEWWDQIPNPARSRLVAIIIQDAQGSQWEKRSRGQ
EPAKCPHWKNCLTKLLPCFLEHNMKRDEDPHKAAKEMPFQGSGKSAWCP
VEISKTVLWPESISVVRCVELFEAPVECEEEEEVEEEKGSFCASPESSR
DDFQEGREGIVARLTESLFLDLLGEENGGFCQQDMGESCLLPPSGSTSA
HMPWDEFPSAGPKEAPPWGKEQPLHLEPSPPASPTQSPDNLTCTETPLV
IAGNPAYRSFSNSLSQSPCPRELGPDPLLARHLEEVEPEMPCVPQLSEP
TTVPQPEPETWEQILRRNVLQHGAAAAPVSAPTSGYQEFVHAVEQGGTQ
ASAVVGLGPPGEAGYKAFSSLLASSAVSPEKCGFGASSGEEGYKPFQDL
IPGCPGDPAPVPVPLFTFGLDREPPRSPQSSHLPSSSPEHLGLEPGEKV
EDMPKPPLPQEQATDPLVDSLGSGIVYSALTCHLCGHLKQCHGQEDGGQ
TPVMASPCCGCCCGDRSSPPTTPLRAPDPSPGGVPLEASLCPASLAPSG
ISEKSKSSSSFHPAPGNAQSSSQTPKIVNFVSVGPTYMRVS
SEQ ID NO: 8
MCVGARRLGRGPCAALLLLGLGLSTVTGLHCVGDTYPSNDRCCHECRPG
NGMVSRCSRSQNTVCRPCGPGFYNDVVSSKPCKPCTWCNLRSGSERKQL
CTATQDTVCRCRAGTQPLDSYKPGVDCAPCPPGHFSPGDNQACKPWTNC
TLAGKHTLQPASNSSDAICEDRDPPATQPQETQGPPARPITVQPTEAWP
RTSQGPSTRPVEVPGGRAVAAILGLGLVLGLLGPLAILLALYLLRRDQR
LPPDAHKPPGGGSFRTPIQEEQADAHSTLAKI
```

1. A bispecific antigen-binding molecule comprising a first antigen binding domain (B1) which is an IL-13 or IL-13R antigen binding domain and a second antigen binding domain (B2) which is an OX40L or OX40 antigen binding domain and wherein the bispecific antigen-binding molecule specifically binds to both (i) IL-13 or IL-13R and (ii) OX40L or OX40.

2. The bispecific antigen-binding molecule of claim 1, which antagonises both IL-13 signalling from IL-13R and OX40L signalling from OX40.

3. The bispecific antigen-binding molecule of claim 1 wherein:
i) B1 specifically binds to IL-13 and B2 specifically binds to OX40L;
ii) B1 specifically binds to IL-13R and B2 specifically binds to OX40L;
iii) B1 specifically binds to IL-13 and B2 specifically binds to OX40; or
iv) B1 specifically binds to IL-13R and B2 specifically binds to OX40.

4. The bispecific antigen-binding molecule according to claim 1, wherein the IL-13 or IL-13R antigen binding domain comprises an antibody or antigen binding fragment thereof, and wherein the antibody is a chimeric, humanized, or human antibody.

5. The bispecific antigen-binding molecule according to claim 1, wherein the OX40L or OX40 antigen binding domain comprises an antibody or antigen binding fragment thereof, and wherein the antibody is a chimeric, humanized, or human antibody.

6. The bispecific antigen-binding molecule according to claim 1, wherein the bispecific antigen binding molecule is a bispecific antibody or an antigen-binding fragment thereof, and optionally wherein the bispecific antibody is a monoclonal antibody and/or is a chimeric, humanized, or human antibody.

7. The bispecific antigen-binding molecule according to claim 6 wherein: (a) the bispecific antibody comprises an IgG1, IgG2, IgG3 or IgG4 constant region, optionally a human IgG1, IgG2, IgG3 or IgG4 constant region; and/or (b) the bispecific antibody is: i) an IgG-like bispecific antibody; or ii) a non-IgG like bispecific antibody.

8. The bispecific antigen-binding molecule according to claim 7 wherein the IgG-like bispecific antibody is: i) a symmetric IgG-like bispecific antibody; or ii) a non-symmetric IgG-like bispecific antibody.

9. The bispecific antigen-binding molecule according to claim 6 wherein the bispecific antibody comprises variable domains of an antibody and T cell receptor (TCR) constant regions, wherein the TCR constant regions are capable of forming a dimer comprising at least one non-native interchain bond.

10. A pharmaceutical composition comprising the bispecific antigen-binding molecule of claim 1 and a pharmaceutically acceptable carrier.

11. A method of treating a disease or condition in a patient, wherein:
(i) the disease or condition is associated with or mediated by IL-13 and/or OX40L, and wherein the method comprises administering to the patient a bispecific antigen-binding molecule according to claim 1.

12. The method according to claim 12, wherein the disease or condition is selected from the group consisting of: a dermatological disease asthma, allergic diseases, cardiovascular diseases, atherosclerosis, musculoskeletal diseases, COPD, age-related macular degeneration, periodontitis uveitis, cancer, inflammatory bowel disease, fibrosis, scleroderma, and eosinophilic esophagitis.

13. The method according to claim 12 wherein: (i) the disease or condition is a dermatological disease; (ii) the bispecific antigen binding molecule is administered to the patient by injection; and/or (iii) the patient is a human patient.

14. The method according to claim 12, wherein the method further comprises administering an additional medication and/or carrying out a separate parallel treatment method for the treatment of the disease or condition.

15. (canceled)

16. (canceled)

17. The bispecific antigen-binding molecule according to claim 8, wherein the symmetric IgG-like bispecific antibody is a dual-variable domain immunoglobulin (DVD-Ig) bispecific antibody.


SEQUENCE LISTING (4 sequences)

SEQ ID NO: 1 - protein, 146 aa, Homo sapiens [sequence omitted]

SEQ ID NO: 2 - protein, 111 aa, Homo sapiens
PVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 3 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 4 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL